(19) United States
(12) Patent Application Publication
(10) Pub. No.: US 2007/0036867 A1
(43) Pub. Date: Feb. 15, 2007

(54) Controlled and Sustained Gene Transfer Mediated by Thiol-Modified Polymers

(75) Inventors: Shyam S. Mohapatra, Tampa, FL (US); Dong-Won Lee, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/419,878

(22) Filed: May 23, 2006

Related U.S. Application Data
(60) Provisional application No. 60/683,709, filed on May 23, 2005.

(51) Int. Cl. A61K 48/00; A61K 31/722; A61K 9/14

(52) U.S. Cl. 424/489; 514/44; 514/55; 977/906

(57) ABSTRACT

A delivery system employing nanoparticles of thiolated chitosan which complex with nucleic acids or other drugs. The system utilizes an approximately 33 kDa thiol-modified chitosan derivative resulting in highly effective gene delivery. Thiolation of chitosan resulted in reduced density of positive charges and DNA binding capacity. However, thiolated chitosan carrying a plasmid DNA expressing a green fluorescent protein (GFP) showed significantly higher GFP expression in various cell lines and in vivo in mice. Sustained delivery of plasmid DNA from thiolated chitosan was achieved by crosslinking thiolated chitosan/plasmid DNA nanocomplexes through inter- as well as intramolecular disulfide bonds under the physiological conditions. Thiolated chitosan nanoparticles have a great potential for gene therapy and tissue engineering.

### CROSS REFERENCE TO RELATED APPLICATIONS

This application claims priority to currently pending U.S. Provisional Patent Application 60/683,709, entitled, “A Method of Nanoparticle-Mediated Gene Transfer”, filed May 23, 2005, the contents of which are herein incorporated by reference.

### STATEMENT OF GOVERNMENT INTEREST

This invention was made with Government support under Grant No. 5RO1HL71101-O1A2 awarded by the National Institutes of Health. The Government has certain rights in the invention.

### FIELD OF INVENTION

This invention relates to gene delivery systems. More specifically, this invention relates to gene delivery systems using nanoparticles of thiolated chitosan providing enhanced delivery and sustained release of encapsulated DNA.

### BACKGROUND OF THE INVENTION

Advances in genomics and proteomics have led to the discovery of numerous gene targets and the use of gene therapy for treatment of diverse human diseases. Although both viral or non-viral gene delivery systems are under investigation, virus-based gene therapy is limited by concerns about endogenous virus recombinations, oncogenic effects and immunological reactions. The non-viral, polymer- or lipid-based gene delivery agents such as polyamidoamine, polyethyleneimine, poly-L-lysine and poly (lactic-co-glycolic acid) (PLGA) copolymers, offer several advantages, including ease of production and reduced risk of immunogenicity, but their use has been limited by their relatively low transfection efficiency, non-degradability and potential toxicity.

We and others have used chitosan, a linear copolymer of N-acetyl-D-glucosamine and D-glucosamine linked by glucosidic linkages, as a vehicle for in vivo therapeutic transfer of genes and siRNAs. Chitosan has emerged as a promising candidate for gene delivery because of biocompatibility, biodegradability, favorable physicochemical properties and ease of chemical modification. The presence of positive charges from amine groups makes chitosan suitable for modification of its physicochemical and biological properties, and enables it to transport the plasmid DNA (pDNA) into cells via endocytosis and membrane destability. Most studies to date have used high molecular weight chitosan (100˜400 kDa), which exhibits aggregation, low solubility under physiological conditions, high viscosity at concentration used for in vivo delivery and slow dissociation or degradation. However, recently a second generation chitosan with molecular weight up to 50 kDa has been isolated and characterized. Chitosans less than 10 kDa, also known as oligo-chitosans have been described to form weak complexes with pDNA, resulting in physically unstable polyplexes with low transfection efficiency.

### SUMMARY OF INVENTION

A delivery system employing nanoparticles of thiolated chitosan which complex with nucleic acids or other drugs. The system utilizes an approximately 33 kDa thiol-modified chitosan derivative resulting in highly effective gene delivery. Thiolation of chitosan resulted in reduced density of positive charges and DNA binding capacity. However, thiolated chitosan carrying a plasmid DNA expressing a green fluorescent protein (GFP) showed significantly higher GFP expression in various cell lines and in vivo in mice. Sustained delivery of plasmid DNA from thiolated chitosan was achieved by crosslinking thiolated chitosan/plasmid DNA nanocomplexes through inter- as well as intramolecular disulfide bonds under the physiological conditions. Thiolated chitosan nanoparticles have a great potential for gene therapy and tissue engineering.

In accordance with the present invention there is provided a drug delivery system utilizing a thiolated chitosan nanoparticle having thiol groups that are cross-linked. In certain embodiments the thiol groups are cross-linked by oxidation. In advantageous embodiments the oxidized thiol groups are linked in an oxidation reaction having a duration of about 12 or less hours. In alternative embodiments the thiol groups are cross-linked by addition of one or more chemical reagents. Choice of the particular reagent chosen for the cross-linking can affect the thiol groups cross-linked, the degree of cross-linking and the reversibility of cross-linking. Therefore, by varying these parameters, the release pattern can be tailored to the particular needs of the application. In a particularly advantageous embodiment the cross-linking of thiol groups is adapted to provide sustained release of one or more drugs.

In certain embodiments the thiolated chitosan particles range in molecular weight from about 10 kDa to about 100 kDa. In particularly advantageous embodiments the thiolated chitosan particles have a molecular weight of about 33 kDa. In certain embodiments the thiolated chitosan particles are less than about 300 nm. In certain embodiments the thiolated chitosan particles have a deacylation of about 90%.

The present invention also provides a nucleic acid delivery system utilizing a thiolated chitosan nanoparticle. In certain embodiments the nucleic acid delivery system includes a nucleic acid molecule in association with the thiolated chitosan. In certain specific embodiments the weight ratio of thiolated chitosan to nucleic acid is about 1:1 to about 10:1. In yet further specific embodiments the weight ratio of thiolated chitosan to nucleic acid is about 5:1 to about 10:1. In particularly advantageous embodiments the weight ratio of thiolated chitosan to nucleic acid is about 5:1.

The nucleic acid delivery system can further include thiolated chitosan nanoparticles that are cross-linked. The cross-linking of thiol groups can be adapted to provide sustained release of one or more drugs. In certain embodiments the thiol groups are crosslinked by oxidation. In advantageous embodiments the oxidized thiol groups are linked in an oxidation reaction having a duration of about 12 or less hours. In alternative embodiments the thiol groups are cross-linked by addition of one or more chemical reagents.

In certain embodiments the thiolated chitosan particles range in molecular weight from about 10 kDa to about 100 kDa. In particularly advantageous embodiments the thiolated chitosan particles have a molecular weight of about 33 kDa. In certain embodiments the thiolated chitosan particles are less than about 300 nm. In certain embodiments the thiolated chitosan particles have a deacylation of about 90%.

The present invention further provides a method of delivering a nucleic acid to a cell. The method includes the steps of providing a thiolated chitosan nanoparticle, providing a nucleic acid of interest, combining the thiolated chitosan nanoparticle and the nucleic acid of interest under conditions sufficient to form nucleic acid-chitosan complexes and contacting a target cell with the nucleic acid-thiolated chitosan complex. The method can further include the step of crosslinking the thiol residues of the thiolated chitosan nanoparticles. In a particularly advantageous embodiment the cross-linking of thiol groups is adapted to provide sustained release of one or more nucleic acids. In certain embodiments the thiol groups are crosslinked by oxidation. In advantageous embodiments the oxidized thiol groups are linked in an oxidation reaction having a duration of about 12 or less hours. In alternative embodiments the thiol groups are cross-linked by addition of one or more chemical reagents. The crosslinking step can be performed before the step of combining the thiolated chitosan nanoparticle and the nucleic acid of interest.

The present invention further provides a method of delivering a drug to a cell. The method includes the steps of providing one or more thiolated chitosan nanoparticles, crosslinking the thiol residues of the one or more thiolated chitosan nanoparticles, providing a drug of interest and combining the thiolated chitosan nanoparticles and the drug of interest under conditions sufficient to form drug-thiolated chitosan complexes and contacting a target cell with the drug-crosslinked thiolated chitosan complex. In a particularly advantageous embodiment the cross-linking of thiol groups is adapted to provide sustained release of one or more drugs. In certain embodiments the thiol groups are crosslinked by oxidation. In advantageous embodiments the oxidized thiol groups are linked in an oxidation reaction having a duration of about 12 or less hours. In alternative embodiments the thiol groups are cross-linked by addition of one or more chemical reagents. The crosslinking step can be performed before the step of combining the thiolated chitosan nanoparticle and the drug of interest.

### BRIEF DESCRIPTION OF THE DRAWINGS

For a fuller understanding of the invention, reference should be made to the following detailed description, taken in connection with the accompanying drawings, in which:

FIG. 1 illustrates aspects of thiolated chitosan particles. (a) Chemical structure of chitosan-thioglycolic acid conjugates and intermolecular disulfide bonding. (b) Decrease of the thiol group content within CSH60 and CSH360. Conjugates were dissolved in demineralized water at a final concentration of 0.2% and pH was adjusted to 6.0 with 100 mM acetate buffer. The samples were incubated at 37° C. The amount of remaining thiol groups was determined with Ellman's reagent. Indicated values are means (±S.D.) of at least three experiments. (c) TEM micrograph of CSH360/pDNA nanocomplexes at a weight ratio 5:1.

FIG. 2 illustrates properties of cross-linked thiolated chitosan relative to unmodified chitosan and thiolated chitosan. (a) Agarose gel electrophoresis for DNase I protection assay. (b) Release profiles of pDNA from chitosan/pDNA nanocomplexes. CSH360 was complexed with pDNA and then assessed before and after incubation to crosslink thiolated chitosan in the nanocomplexes with pDNA.

FIG. 3 illustrates the effect of weight ratio of chitosan to pDNA on transfection efficiency of chitosan.

FIG. 4 illustrates the increase in transfection efficiency achieved using thiolated or cross-linked, thiolated chitosan as compared to unmodified particles. (a) Representative flow cytometric analysis of GFP-expressing cells 60 h after transfection at a weight ratio of 5:1. (b) Kinetics of gene expression. Transfection was performed with >50% cell confluency. Indicated values are means (±S.D.) of three experiments. *P<0.01 relative to unmodified chitosan at the same time point.

FIG. 5 illustrates the enhanced transfection efficiency achieved with thiolated chitosan as compared to unmodified chitosan. The comparison of transfection efficiency in HEp2 and MDCK cells. Indicated values are means (±S.D.) of three experiments.

FIG. 6 illustrates the sustained gene expression by thiolated chitosan/pDNA nanocomples after cross-linking using immunoblotting analysis of green fluorescent protein production due to reporter gene expression. (a) Comparison of GFP expression mediated by Lipofectin, unmodified chitosan and thiolated chitosan (CSH360) at 60 h post-transfection. Control is untransfected cells. (b) GFP expression mediated by CSH360. (c) GFP expression mediated by crosslinked CSH360.

FIG. 7 illustrates the gene expression of GFP pDNA in mouse BAL cells. (a) Micrographs of BAL cells 14 days after intranasal administration. Gray brightfield images were merged with fluorescence using WICF Image J program (NIH, USA). Control is BAL cells from untreated mice. (b) The level of gene expression in BAL cells. Four different areas of each slide were examined and gene expression level was calculated by counting the number of total cells and GFP expressing cells. *P<0.01 relative to unmodified and thiolated chitosan at 14 days post-intranasal administration (n=4).

### DETAILED DESCRIPTION OF THE PREFERRED EMBODIMENT

Nanoparticles of chitosan, a natural biodegradable polymer, have been investigated for gene delivery. However, the utility of high molecular weight chitosan has been limited by its low water solubility under physiological conditions, aggregation, high viscosity at concentrations used for in vivo delivery and low transfection efficiency. Herein we report on a highly effective gene delivery system utilizing a 33 kDa thiol-modified chitosan derivative. Thiolation of chitosan resulted in reduced density of positive charges and DNA binding capacity. However, thiolated chitosan carrying a plasmid DNA expressing a green fluorescent protein (GFP) showed significantly higher GFP expression in various cell lines and in vivo in mice. Sustained delivery of plasmid DNA from thiolated chitosan was achieved by crosslinking thiolated chitosan/plasmid DNA nanocomplexes through inter- as well as intramolecular disulfide bonds under the physiological conditions. Thus, thiolated chitosan nanoparticles have a great potential for gene therapy and tissue engineering.

We have been investigating the potential for enhancing gene transfer by thiol modification of chitosan. Thiolated chitosan appears to possess significantly improved mucoadhesiveness and to enhance the permeability properties of drugs, but the potential of thiolated chitosans for gene transfer has not been studied. Since thiolated chitosan forms inter- as well as intramolecular disulfide bonds upon oxidation, it was reasoned that thiolation may allow crosslinking of chitosan, which, in turn may allow slow sustained release of pDNA. To test this, a 33 kDa chitosan with a high degree of deacetylation (>90%) was characterized and tested for enhanced and sustained gene delivery and expression in the absence or presence of thiolation with or without crosslinking. The results indicate that thiolated chitosan forms nanocomplexes with pDNA encoding the reporter gene for the green fluorescence protein (GFP) and allows sustained gene delivery and expression of GFP both in vitro and in vivo.

Results

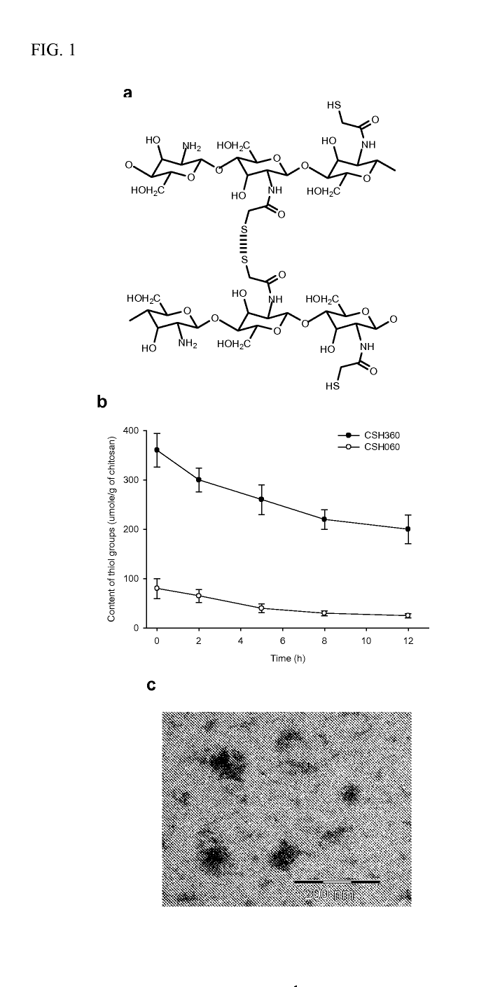

Characterization and physicochemical properties of thiolated chitosan. To synthesize and characterize the nanocomplexes of chitosan and pDNA, 33 kDa chitosan was used as a starting material. To graft thiol groups on chitosan, the primary amine groups of chitosan were utilized, as shown in FIG. 1a. Water soluble EDAC (1-ethyl-3-(3-dimethylaminopropyl) carbodiimide hydrochloride) reacted with carboxyl groups of thioglycolic acid to form active ester intermediates which reacted with free amine groups of chitosan to form amide bonds. Lyophilized thiolated chitosan appears as a white fibrous powder easily soluble in water. From the spectrophotometric assay using Ellman's reagent, 5,5′-dithiobis(2-nitrobenzoic acid), the content of thiol groups conjugated to chitosan molecules was determined to be equivalent to 60.0±10.0 or 360±34 μmole per 1 gram of chitosan, depending on the ratio between chitosan and thioglycolic acid. The preparations were referred to as CSH060 and CSH360, respectively. It was estimated that ˜2.5 and ˜12.5 thiol groups were grafted to each CSH60 and CSH360 molecule. The content of thiol groups decreased constantly during an incubation at 37° C. at pH 6.0 (FIG. 1b). CSH360 and CSH060 exhibited a reduction in thiol group content by 33% and 40%, respectively, indicating the formation of inter- as well as intramolecular disulfide bonds.

Physicochemical properties of unmodified chitosan/pDNA nanocomplexes with various weight ratios were characterized in terms of size and zeta potential. The nanocomplexes ranged from 75 to 120 nm in diameter and from +2.3 to +19.7 mV in zeta potential, which was directly related to the ratio of chitosan to pDNA and hence the surface charge (Table 1). The nanocomplexes of thiolated chitosans with pDNA showed a reduction in zeta potential (Table 2), but the size remained similar to unmodified chitosan nanocomplexes, i.e., about 120 nm as determined by transmission electron microscopy (FIG. 1c). CSH360/pDNA nanocomplexes were incubated at 37° C. for 12 h to oxidize thiol groups to crosslink thiolated chitosan through the formation of inter- as well as intramolecular disulfide bonds. Crosslinking of thiolated chitosan in the nanocomplexes increased the particle size to some extent; however, zeta potential was not altered. The biocompatibility of chitosan and its derivatives was evaluated using human embryonic kidney (HEK) 293 cells according to a standard methyl thiazole tetrazolium (MTT) cytotoxicity assay. Unmodified chitosan incubated for 6 or 12 h with HEK 293 cells exhibited no cytotoxicity at all weight ratios (data not shown). Both thiolation and crosslinking showed no influence on cell viability.

TABLE 1
Characteristics of nanocomplexes of unmodified
chitosan with pDNA expressing GFP.

| Table 1 | | | |
|---|---|---|---|
| Weight ratio | Charge | | |
| (Chitosan/pDNA) | ratio (+/−) | Particle size (nm) | Zeta potential (mV) |
| 1:1 | 1.8:1 | 75 ± 4 | 2.3 ± 0.3 |
| 2.5:1 | 4.5:1 | 92 ± 4 | 7.6 ± 1.1 |
| 5:1 | 9.0:1 | 120 ± 7 | 19.7 ± 3.9 |

Particle size and zeta potential of nanocomplexes were measured at pH 6.2 by dynamic light scattering and electrophoretic light scattering. Indicated values are means (±S.D.) of three experiments.

TABLE 2
Characteristics of nanocomplexes with pDNA and
transfection efficiency in HEK 293 cells by flow
cytometry at a chitosan:pDNA ratio of 5:1 (wt/wt)

| | Content of | | | Transfection |
|---|---|---|---|---|
| | thiol groups | Particle | Zeta | efficiency |
| | (μ mole/g of | size | potential | (%) at |
| Table 2 | chitosan) | (nm) | (mV) | pH 7.0 |
| Lipofectin | N/A | N/A | N/A | 16.4 ± 2.1 |
| Unmodified | 0 | 120 ± 7 | 19.7 ± 3.9 | 5.8 ± 1.1 |
| chitosan | | | | |
| Thiolated | 360 ± 34 | 113 ± 7 | 12.5 ± 2.1 | 31.2 ± 4.3 |
| chitosan | | | | |
| CSH360 | | | | |
| Thiolated | 60 ± 10 | 103 ± 10 | 15.3 ± 3.3 | 12.3 ± 2.3 |
| chitosan CSH060 | | | | |
| Crosslinked | 200 ± 20.1 | 220 ± 16 | 7.3 ± 2.8 | 18.1 ± 2.8 |
| CSH360 | | | | |

Transfection efficiency was measured at 60 h post-transfection. Indicated values are means (±S.D.) of three experiments.

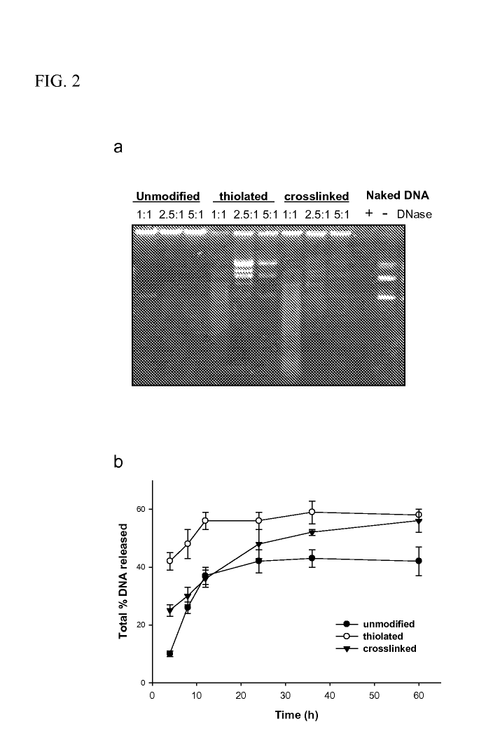

Thiolation protects DNA and allows slow DNA release. To examine the effect of thiolation and crosslinking of thiolated chitosan on pDNA binding capacity and protection ability, chitosan/pDNA nanocomplexes were treated with DNase I and dissociated by the addition of heparin. Unmodified chitosan protected pDNA in the complexes and retained pDNA completely at all weight ratios (1:1˜5:1). Thiolated chitosan (CSH360) exhibited effective physical stability and protection against DNase I digestion at a weight ratio≧2.5:1 (FIG. 2a). After the incubation of CSH360/pDNA nanocomplexes at 37° C. for 12 h to crosslink the thiolated chitosan, the subsequent crosslinked CSH360 at a weight ratio of 1:1 partially protected and retained DNA, while non-crosslinked CSH360 allowed digestion and release of pDNA. Very effective complexation and protection against DNase I degradation were observed with crosslinked CSH360 at a weight ratio of 5:1.

To investigate the in vitro pDNA release profile, chitosan/pDNA nanocomplexes were incubated in a transfection medium at 37° C. After various periods of incubation time, chitosan/pDNA nanocomplexes were centrifuged and the content of pDNA released was determined (FIG. 2b). From the formulations made with unmodified chitosan, only a small fraction of pDNA was released during the first 4 h, followed by a rapid release by 12 h post-incubation. In contrast, an initial pDNA release was observed for thiolated chitosan (CSH360) and the majority (>55%) of pDNA was released by 12 h. However, not all pDNA was released from the electrostatic nanocomplexes because of effective complexation at a weight ratio of 5:1. Crosslinking of CSH360 drastically affected the pDNA release kinetics. pDNA was continuously released from the formulation with crosslinked CSH360 for at least 60 h.

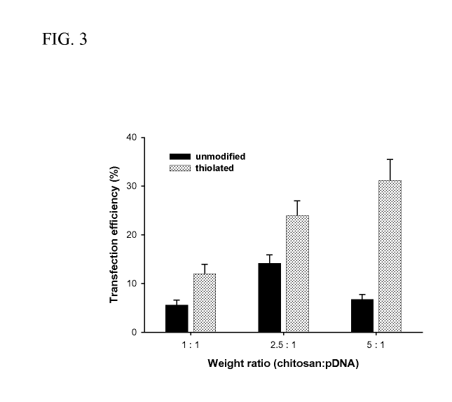

Thiolation enhances transfection of cultured cells. To determine the in vitro transfection efficiency of thiolated chitosan and crosslinked thiolated chitosan, HEK 293 cells were transfected with chitosan nanocomplexes containing pDNA encoding GFP in transfection medium (pH 7.0). GFP-positive cells were scored by flow cytometry. For unmodified chitosan with 90% deacetylation, the highest transfection efficiency was obtained at a weight ratio of 2.5:1. In contrast, for thiolated chitosan (CSH360), the transfection efficiency increased with increasing weight ratio from 1:1 to 5:1 (FIG. 3). However, a further increase in a weight ratio to 10:1 did not increase the transfection efficiency. CSH360 exhibited higher transfection efficiency than the unmodified chitosan or a liposomal transfection reagent, Lipofectin (Invitrogen, USA). It was also found that thiolated chitosan (CSH360) with a higher thiol group content exhibited a higher transfection efficiency (Table 2). To further investigate the effect of thiol group content on gene transfer, thiol groups of CSH360 were oxidized to decrease the thiol group content and then mixed with pDNA to form nanocomplexes. After oxidation for 12 h, CSH360 exhibited reduced thiol group content and a subsequent reduction in the transfection efficiency (Table 2).

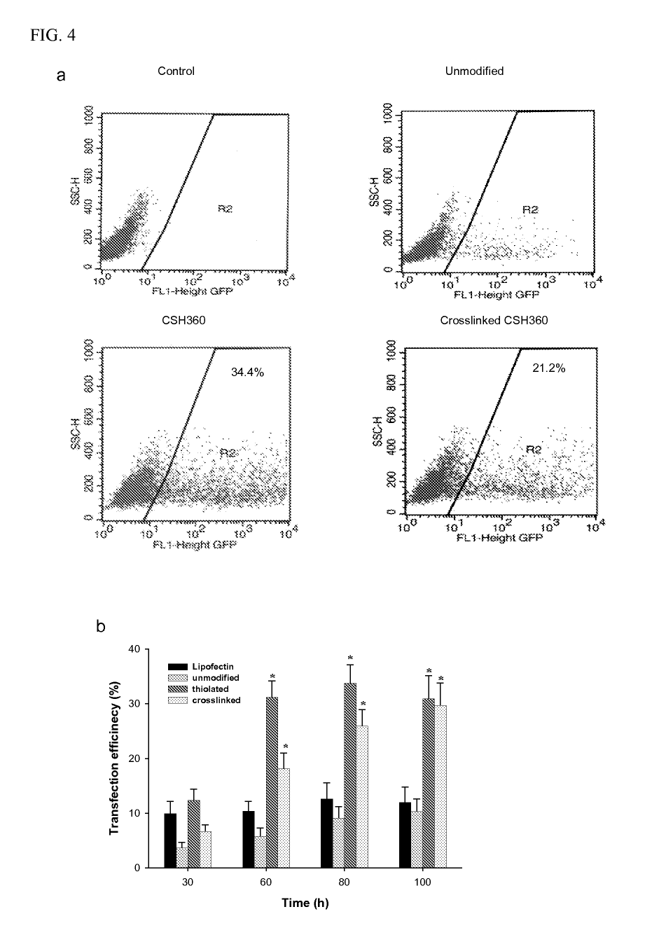

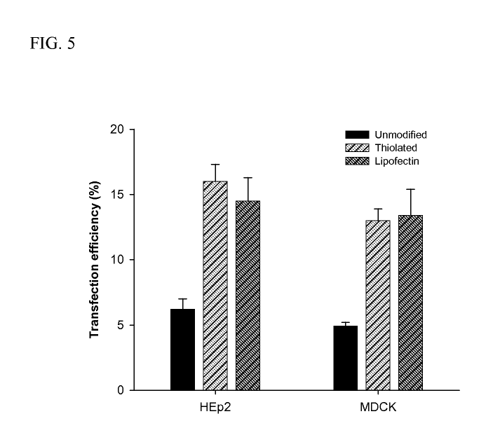

To test whether thiolation and crosslinking of thiolated chitosan/pDNA nanocomplexes affect the pDNA release profile and gene expression level, the time course of transfection was examined using HEK293 cells and the percentage of transfection was monitored for 4 days after transfection. A significant (P<0.01) increase in transfection efficiency was found with cells transfected with CSH360/pDNA and crosslinked CSH360/pDNA nanocomplexes compared to unmodified chitosan/pDNA nanocomplexes at 60 h post-transfection (FIG. 4). CSH360/pDNA nanocomplexes showed rapid enhanced gene expression by 60 h and reached a plateau soon after. In contrast, crosslinked CSH360/pDNA nanocomplexes exhibited a gradual increase in gene expression for 4 days. To confirm the superior transfection efficiency of CSH360, transfection was performed using two other cell lines, HEp-2 and MDCK. Transfection efficiency with each of the cell lines studied was lower than HEK293, but thiolated chitosan exhibited a higher transfection efficiency than unmodified chitosan (FIG. 5).

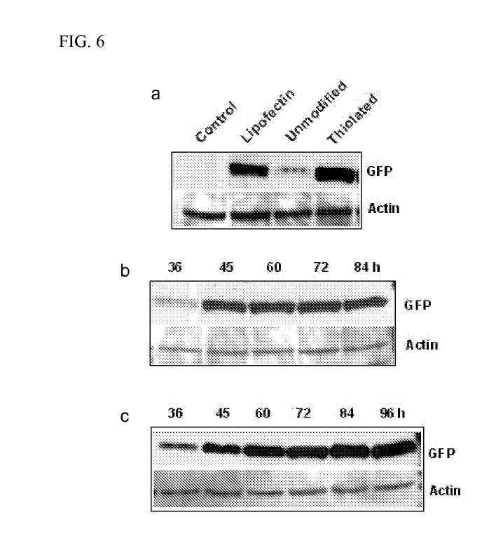

To further support sustained gene expression by thiolated chitosan/pDNA nanocomplexes after crosslinking, we extracted green fluorescent proteins synthesized in transfected cells to examine the level of gene expression using an immunoblotting assay. Thiolated chitosan (CSH360) induced more gene expression than Lipofectin and unmodified chitosan at an identical time point (FIG. 6a). While CSH360 showed rapid gene expression, crosslinked CSH360 showed a steady increase in gene expression during an observation period of 96 h (FIG. 6b-c). The results suggest that thiolation of chitosan increases the transfection efficiency and that sustained gene expression can be achieved by crosslinking the thiolated chitosan in the nanocomplexes with pDNA.

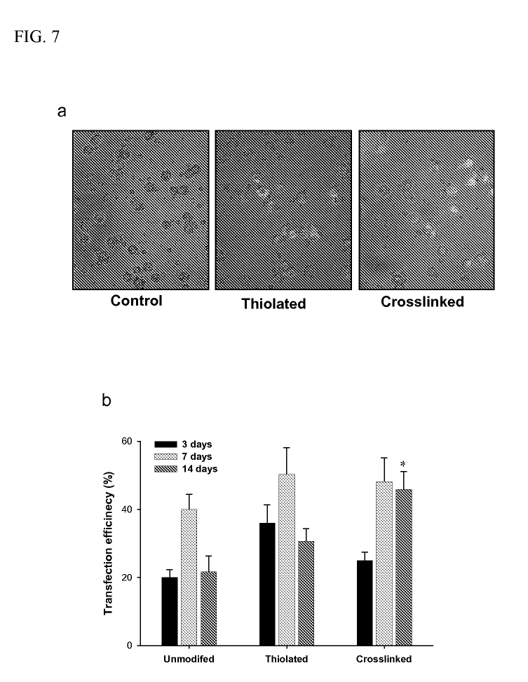

Thiolated chitosan enhances in vivo gene delivery. To investigate the in vivo gene transfer potential of CSH360 and crosslinked CSH360, we utilized the nasal cavity for the route of nanoparticle delivery because of its greater permeability than other administration routes and avoidance of first-pass metabolism in the liver.²⁵ The in vivo transfection efficiency of thiolated chitosan (CSH360) was studied after intranasal administration of pDNA to mice by observing the cells in bronchoalveolar lavage (BAL) fluid (FIG. 7). At 3 days post-intranasal administration, CSH360/pDNA nanocomplexes yielded more gene expression than that induced by unmodified or crosslinked CSH360. Crosslinked CSH360/pDNA nanocomplexes exhibited increased gene expression after 7 days in comparison to that observed after 3 days. At 14 days post-intranasal administration, crosslinked CSH360 mediated more gene expression than unmodified and CSH360. These observations suggest that thiolation of chitosan and subsequent crosslinking enhance gene delivery potential and mediate sustained gene expression.

Discussion

Chitosan appears to be one of the most promising carrier of genes because of its many advantages including biodegradability, biocompatibility, non-toxicity, non-immunogenicity, and wound-healing properties. The most important limitation of chitosan as a gene carrier, which has limited its clinical potential, is its low cellular transfection efficiency. The results of our studies in this report demonstrate that thiolated chitosan represents an advanced generation of nanocomplexes that exhibit enhanced gene expression and, upon crosslinking, can generate a slow, sustained release of pDNA and gene expression both in cultured cells and in mice.

Both high and low molecular weight chitosan nanoparticles have their advantages and disadvantages. The chemical modification of chitosan alters mainly the degree of deacetylation. It was also reported that at constant molecular weight, the reduction of the degree of deacetylation decreased the zeta potential and DNA binding capacity, subsequently leading to a reduction in transfection efficiency. A moderate molecular weight 33 kDa chitosan with 90% deacetylation was chosen to develop chitosan/pDNA nanocomplexes. Thiolation of chitosan was expected to decrease positive charge density resulting in a lower zeta potential, and hence decreased transfection efficiency. Thus, the finding that thiolated chitosan exhibits a higher in vitro and in vivo transfection efficiency is contrary to the commonly accepted notion that a higher zeta potential is required for increased transfectability. In addition, thiolated chitosan exhibited reduced transfection efficiency after the formation of intra- as well as intermolecular disulfide bonds, despite the unchanged zeta potential, which suggests that enhanced gene transfer of thiolated chitosan is mediated by the introduced thiol groups. The mechanism underlying increased transfectability of thiolated chitosan is unclear. It might be due to increased mucoadhesion and cell permeation properties, as suggested previously. Unmodified chitosan formed extremely stable complexes with pDNA and delayed the pDNA release at weight ratios (>2.5:1), leading to low transfection efficiency. This is in good agreement with previous studies, which showed that the high physical stability of chitosan is a major rate-limiting step for the intercellular release of pDNA from complexes. One of the possible explanations for the enhanced gene delivery of thiolated chitosan is that thiolation of chitosan reduces the positive charge density and pDNA complexing capacity, resulting in more rapid pDNA release. An alternative chemical reaction of chitosan was performed with butanoic anhydride to answer the question of whether partial neutralization of positive charges increases the transfection efficiency of chitosan. Butanoyl chitosan exhibited reduced surface charges and less DNA binding capability which can result in easy and rapid DNA release. However, butanoyl chitosan showed less transfection efficiency than unmodified chitosan (supplementary material). From this observation, it can be concluded that the enhanced gene transfer capability of thiolated chitosan is not only from the reduced DNA binding capability by partial neutralization of positive charges, but also from increased mucoadhesion and cell permeation properties by introduced thiol groups. Alternatively, transfection efficiency of crosslinked thiolated chitosan might result from the thiolation-endowed physical stability of chitosan/pDNA nanocomplexes by crosslinking of thiolated chitosan through the inter- as well as intramolecular disulfide bonding, and protection of complexed pDNA from nucleases, as shown by the results of this study. The delay of pDNA release can be explained by the rationale that crosslinking of thiolated chitosan results in effective entrapping and/or immobilizing pDNA.

A major finding of these studies is that thiolated chitosan supports significantly enhanced transfection in cells, notably higher than a commercial transfection reagent, Lipofectin. It is noteworthy that transfection is highly pH-dependent, irrespective of the transfection agent. Lipofectin has extremely high transfection efficiency (>45%) at pH 7.5, but exhibits significantly diminished transfection efficiency at pH 7.0. On the other hand, both unmodified and thiolated chitosans showed significantly higher transfection efficiency at pH 7.0 than at pH 7.5. This observation is in good accordance with previous studies, in which chitosan of 40 kDa showed higher transfection efficiency at pH 7.0 than at pH 7.5. Ishii et al. reported that the effect of pH on the transfection capability of chitosan is presumably due to the fact that chitosan has slightly positive charges at pH 7.0. Because of protonation of amine groups, chitosan, through electrostatic interaction, forms more stable complexes with DNA and the cell membrane, thus perturbing the cell membrane bilayers and resulting in higher transfection.

One of the most important feature of thiolated chitosan is its intrinsic ability to readily oxidize its thiol groups to form inter- as well as intramolecular disulfide bonds. The results show that crosslinking of thiolated chitosan promotes extended release of pDNA both in vitro in cultured cells and in mice. Most likely, the thiolation feature allows sustained gene expression over several days, which is key to achieving the therapeutic efficacy of DNA delivery and expression of gene products. The mechanism underlying this is unclear. It is possible that not all thiol groups participate in inter- and intramolecular disulfide bonding. Thus, thiol groups located close to each other form disulfide bonds readily and form the networked structure through the crosslinking. The remaining thiol groups cannot oxidize without neighboring thiol groups and play a role in mucoadhesion and permeability enhancement. This rationale is supported by the in vitro pDNA release test and the slow and continuous gene expression both in vivo and in vitro.

The present work reports, for the first time, chitosan-based nanocomplexes for sustained gene delivery, adding this to a number of sustained DNA delivery systems including poly(lactide-co-glycolide) (PLG) matrices, collagen sponges, PLGA emulsion coating, PLGA nanoparticles, poly(ethylene-co-vinyl acetate) (EVAc) disks, gelatin nanospheres, and Pluronic hydrogels. The major limitations of these sustained delivery systems are the tedious procedures and the use of harsh chemical reagents which are toxic and difficult to remove completely. In remarked contrast to other sustained release microspheres or nanoparticles, the crosslinking of thiolated chitosan nanocomplexes for sustained gene delivery of DNA is accomplished under very mild conditions without any chemical crosslinking agent. An ideal non-viral gene delivery system must have well-defined physicochemical characteristics and the following properties, including ease of assembly with DNA, stabilization of DNA before and after cell uptake, the capability of endocytic pathways, and adjustable expression of the therapeutic level of proteins over time. The thiolation and crosslinking of thiol groups may help chitosan fulfill many of these requirements.

In conclusion, these results demonstrate that thiolated chitosan forms nanocomplexes with pDNA and exhibits significantly improved gene delivery potential in vitro as well as in vivo. The extended pDNA release and subsequent slow gene expression were achieved by oxidation of introduced thiol groups to crosslink the thiolated chitosan, thus presenting a novel approach with great potential for enhanced and sustained gene delivery.

Materials and Methods

Preparation of thiolated chitosan and plasmid DNA. Chitosan (MW 33 kDa, degree of deacetylation>90%, viscosity 2.8 cps at 0.5% solution in 0.5% acetic acid at 20° C., Taehoon Bio. Korea) of 0.5 g was dissolved in 50 ML of aqueous acetic acid solution (1.0%) to which 400 or 100 μL ofthioglycolic acid (TGA) was added. In order to activate the carboxylic acid moieties of TGA, 0.5 g of 1-ethyl-3-(3-dimethylaminopropyl)carbodiimide hydrochloride (EDAC) was added. The pH of the solution was adjusted to 5.0 using 1 mM NaOH and the chemical reaction was allowed to run at room temperature for 5 h. To eliminate unbound TGA and isolate the conjugated polymers, the reaction mixture was dialyzed (molecular weight cut-off 6 kDa). The chitosan conjugate was lyophilized at −30° C. and stored at 4° C. until further use. The degree of chemical modification of the chitosan-thioglycolic acid conjugate was determined spectrophotometically by measuring thiol groups at room temperature using Ellman's reagent, 5,5′-dithiobis(2-nitrobenzoic acid) at a wavelength of 412 nm. A plasmid (pEGFP-N2, 4.7 kbp, Clontech, USA) containing the human cytomegalovirus promoter (CMV) and enhanced green fluorescent protein gene was amplified in E. Coli and purified using GenElute HP Plasmid Maxprep Kits (Sigma, USA).

Preparation and characterization of chitosan/pDNA nanocomplexes. Chitosan/pDNA nanocomplexes were prepared by mixing chitosan (2 μg/μL) and pDNA (2 μg/μL) solution in phosphate buffer at pH 6.2. The chitosan/DNA charge ratio was determined assuming a molecular weight of plasmid DNA of 325 g/mol and one negative charge per DNA base. Positive charge units were calculated assuming one positive charge per amine group adjusted for the degree of deacetylation of chitosan. The loss of amine groups after thiolation was not considered in the calculation of positive charges of thiolated chitosans. Nanocomplexes of thiolated chitosan (CSH360) with pDNA were incubated at 37° C. for 12 h to oxidize thiol groups to crosslink thiolated chitosan in the nanocomplexes. Particle size and zeta potential of chitosan/pDNA nanocomplexes were measured using a Nicomp380/ZLS (Particle Sizing Systems Inc. CA) at 25° C.

MTT cytotoxicity assay. The evaluation of cytotoxicity of thiolated chitosan was performed by MTT assay using the HEK 293 cell line. Cells were seeded at 5.0×10⁵ cells/well in a 12 well flat-bottomed tissue culture plate and incubated for 24 h. Chitosan/DNA complexes were added and incubated for 6 h at 37° C. The transfection mixture was replaced with 500 μL of serum-free DMEM to which 150 μL MTT solution (2 mg/mL) in PBS was added. After incubation at 37° C. for 4 h, the MTT-containing medium was removed and 750 μL of DMSO was added to dissolve the formazan crystals formed by cells. Cell viability was determined by measuring the absorbance at 570 nm.

Protection against DNase I degradation. pDNA (1 μg) alone or chitosan/pDNA nanocomplexes were prepared in 10 μL of phosphate buffer at pH 6.2 to which 2 μL of DNase I (5U) was added and incubated at 37° C. for 2 h. Then, 5 μL of 100 mM EDTA was added and the mixture was incubated at room temperature for 10 min. After incubation, 10 μL of heparin solution (5 mg/mL) was added to the mixture and incubated at room temperature for 2 h to dissociate the complexes. The integrity of plasmid DNA was examined using the agarose gel retardation assay.

In-vitro DNA release. Chitosan/pDNA complexes were incubated in a transfection medium (DMEM with pH 7.0) at 37° C. After different periods of incubation, chitosan/pDNA complexes were centrifuged at 16,000×g for 30 min and the supernatants were collected to determine the DNA content by measuring the fluorescent intensity after the addition of fluorescent nucleic acid strain (Quanti-iT™ PicoGreen®, Molecular Probes, USA).

In vitro transfection. Transfection medium was prepared by dissolving Dulbecco's Modified Eagles' Medium (Sigma) in sterile water and adjusting pH to 7.0 by adding sodium bicarbonate. HEK 293 cells were seeded in a six-well culture plate at a density of 1×10⁶ cells/well and incubated at 37° C. in a CO₂ incubator for 24 h. The solutions of pDNA and various amount of chitosan were diluted separately in 50 μL of transfection medium. After 5 min, the two solutions were combined, mixed gently and incubated at room temperature for 20 min. Then, 900 μL of transfection medium was added to each tube containing chitosan/pDNA nanocomplexes. The formulations were mixed gently and added to cells. After 8 h incubation, the medium and chitosan/DNA nanocomplexes were replaced with fresh DMEM containing 5 % FBS.

Flow cytometry. To quantify the transfection efficiency of chitosan, transfected cells were harvested and scored for GFP-positive cells by flow cytometry (FACScan, BD Biosciences, USA) with appropriate gating and controls using the green channel FL-1H. A total of 1.5×10⁴ events were counted for each sample and more than 90% of cells were gated for analysis. The percentage of positive events was calculated as the events within the gate divided by total number of events then subtracting percentage of control samples.

Immunoblotting. Proteins were extracted from transfected HEK 293 cells after various periods of incubation times using lysis buffer. Electrophoresis was performed using 40 μg of cell lysate on a 12% polyacrylamide gel and proteins were transferred to PVDF membranes (Bio-Rad, USA). The blot was incubated with a rabbit anti-green fluorescent protein polyclonal antibody (Chemicon, USA) and HRP-conjugated anti-rabbit IgG (Cell Signaling, USA) which is used as a secondary antibody. Immunoblot signals were developed using SuperSignal Ultra chemiluminescent reagent (Pierce, USA).

Examination of bronchoalveolar lavage (BAL) fluid. Plasmid DNA (15 μg) was combined with chitosan solution (2 mg/ml) to form nanocomplexes. These nanocomplexes were then given intranasally to 4-6 week old BALB/c mice (n=4) on day 0. Mice were sacrificed on days 3, 7, 14 days and lungs were lavaged with 500 μL of PBS introduced through the trachea. The BAL fluid was centrifuged, washed with PBS and resuspended in PBS. Aliquots of the cell suspension (150 μL) were applied to slides using cytospin apparatus. Cells were examined under a fluorescent microscope (ECLIPSE TE300 Inverted Microscope, Nikon, Japan) for GFP expression and photography. Fluorescent images were made with a fixed exposure time so that low-intensity auto-fluorescence of BAL cells was not imaged.

### REFERENCES

Agnihotri S A, Mallikarjuna N N, Aminabhavi T M. Recent advances on chitosan-based micro- and nanoparticles in drug delivery. Journal of Controlled Release 2004; 100: 5-28.

Bernkop-Schnurch A, Hornof M, Zoidl T. Thiolated polymers-thiomers: synthesis and in vitro evaluation of chitosan-2-iminothiolane conjugates. International Journal of Pharmaceutics 2003; 260: 229-237.

Bernkop-Schnurch A, Kast C E, Guggi D. Permeation enhancing polymers in oral delivery of hydrophilic macromolecules: thiomer/GSH systems. Journal of Controlled Release 2003; 93: 95-103.

Bernkop-Schnurch A, Guggi D, Pinter Y. Thiolated chitosans: development and in vitro evaluation of a mucoadhesive, permeation enhancing oral drug delivery system. Journal of Controlled Release 2004; 94: 177-186.

Bernkop-Schnurch A, Hornof M, Guggi D. Thiolated chitosans. European Journal of Pharmaceutics and Biopharmaceutics 2004; 57: 9-17.

Cerchiara T, Luppi B, Bigucci F, Zecchi V. Chitosan salts as nasal sustained delivery systems for peptidic drugs. Journal of Pharmacy and Pharmacology 2003; 55: 1623-1627.

Chun K W, Lee J B, Kim S H, Park T G. Controlled release of plasmid DNA from photo-cross-linked pluronic hydrogels. Biomaterials 2005; 26: 3319-3326.

Cohen H et al. Sustained delivery and expression of DNA encapsulated in polymeric nanoparticles. Gene Therapy 2000; 7: 1896-1905.

Fang N, Chan V, Mao H Q, Leong K W. Interactions of phospholipid bilayer with chitosan: effect of molecular weight and pH. Biomacromolecules 2001; 2: 1161-1168.

Hellermann G, Mohapatra S S. Genetic therapy: on the brink of a new future. Genetic Vaccines and Therapy. 2003; 4: 1.

Ii'ina A V, Varlamov V P. Chitosan-based polyelectrolyte complexes: A review. Applied Biochemistry and Microbiology 2005; 41: 5-11.

Ishii T, Okahata Y, Sato T. Mechanism of cell transfection with plasmid/chitosan complexes. Biochimica Et Biophysica Acta-Biomembranes 2001; 1514: 51-64.

Kast C E, Bernkop-Schnurch A. Thiolated polymers—thiomers: development and in vitro evaluation of chitosan-thioglycolic acid conjugates. Biomaterials 2001; 22: 2345-2352.

Kiang T, Wen H, Lim H W, Leong K W. The effect of the degree of chitosan deacetylation on the efficiency of gene transfection. Biomaterials 2004; 25: 5293-5301.

Klugherz B D et al. Gene delivery from a DNA controlled-release stent in porcine coronary arteries. Nature Biotechnology 2000; 18: 1181-1184.

Koping-Hoggard M et al. Chitosan as a nonviral gene delivery system. Structure-property relationships and characteristics compared with polyethylenimine in vitro and after lung administration in vivo. Gene Therapy 2001; 8: 1108-1121.

Koping-Hoggard M et al. Improved chitosan-mediated gene delivery based on easily dissociated chitosan polyplexes of highly defined chitosan oligomers. Gene Therapy 2004; 11: 1441-1452.

Lee D W, Baney R H. Oligochitosan derivatives bearing electron-deficient aromatic rings for adsorption of amitriptyline: Implications for drug detoxification. Biomacromolecules 2004; 5: 1310-1315.

Lee K Y et al. Preparation of chitosan self-aggregates as a gene delivery system. Journal of Controlled Release 1998; 51: 213-220.

Lee M et al. Water-soluble and low molecular weight chitosan-based plasmid DNA delivery. Pharmaceutical Research 2001; 18: 427-431.

Lorenzo-Lamosa M L, Remunan-Lopez C, Vila-Jato J L, Alonso M J. Design of microencapsulated chitosan microspheres for colonic drug delivery. Journal of Controlled Release 1998; 52: 109-118.

Luo D, Saltzman W M. Synthetic DNA delivery systems. Nature Biotechnology 2000; 18: 33-37.

Mansouri S et al. Chitosan-DNA nanoparticles as non-viral vectors in gene therapy: strategies to improve transfection efficacy. European Journal of Pharmaceutics and Biopharmaceutics 2004; 57: 1-8.

Mohapatra S S. Mucosal gene expression vaccine: a novel vaccine strategy for respiratory syncytial virus. Pediatric Infectious Disease Journal 2003; 22: S100-S104.

Nina Langoth H K, Gudrum Schoffmann, Ivo Schmerold, Maximilian Schuh, Sonja Franz, Peter Kurka, Andreas Bernkop-Schnurch. Thiolated Chitosans: Design and In Vivo Evaluation of a Mucoadhesive Buccal Peptide Drug Delivery System. Pharmaceutical Research 2006.

Ravi Kumar M et al. Intranasal gene transfer by chitosan-DNA nanospheres protects BALB/c mice against acute respiratory syncytial virus infection. Human Gene Therapy 2002; 13: 1415-1425.

Ravi Kumar M et al. Cationic poly(lactide-co-glycolide) nanoparticles as efficient in vivo gene transfection agents. Journal of Nanoscience and Nanotechnology 2004; 4: 990-994.

Ravi Kumar M, Hellermann G, Lockey R F, Mohapatra S S. Nanoparticle-mediated gene delivery: state of the art. Expert Opinion on Biological Therapy 2004; 4: 1213-1224.

Ravi Kumar M et al. Cationic silica nanoparticles as gene carriers: Synthesis, characterization and transfection efficiency in vitro and in vivo. Journal of Nanoscience and Nanotechnology 2004; 4: 876-881.

Ravi Kumar M et al. Chitosan IFN-r-pDNA Nanoparticle (CIN) Therapy for Allergic Asthma. Genetic Vaccines Therapy 2003; 1: 3.

Roldo M, Hornof M, Caliceti P, Bernkop-Schnurch A. Mucoadhesive thiolated chitosans as platforms for oral controlled drug delivery: synthesis and in vitro evaluation. European Journal of Pharmaceutics and Biopharmaceutics 2004; 57: 115-121.

Salem A K, Searson P C, Leong K W. Multifunctional nanorods for gene delivery. Nature Materials 2003; 2: 668-671.

Sato T, Ishii T, Okahata Y. In vitro gene delivery mediated by chitosan. Effect of pH, serum, and molecular mass of chitosan on the transfection efficiency. Biomaterials 2001; 22: 2075-2080.

Scherer F et al. Nonviral vector loaded collagen sponges for sustained gene delivery in vitro and in vivo. Journal of Gene Medicine 2002; 4: 634-643.

Shea L D, Smiley E, Bonadio J, Mooney D J. DNA delivery from polymer matrices for tissue engineering. Nature Biotechnology 1999; 17: 551-554.

Shen H, Goldberg E, Saltzman W M. Gene expression and mucosal immune responses after vaginal DNA immunization in mice using a controlled delivery matrix. Journal of Controlled Release 2003; 86: 339-348.

Shen H, Tan J, Saltzman W M. Surface-mediated gene transfer from nanocomposites of controlled texture. Nature Materials 2004; 3: 569-574.

Truong-Le V L, August J T, Leong K W. Controlled gene delivery by DNA-gelatin nanospheres. Human Gene Therapy 1998; 9: 1709-1717.

Wang Y C, Kao S H, Hsieh H J. A chemical surface modification of chitosan by glycoconjugates to enhance the cell-biomaterial interaction. Biomacromolecules 2003; 4: 224-231.

Yamada T et al. Nanoparticles for the delivery of genes and drugs to human hepatocytes. Nature Biotechnology 2003; 21: 885-890.

Yun Y H, Goetz D J, Yellen P, Chen W L. Hyaluronan microspheres for sustained gene delivery and site-specific targeting. Biomaterials 2004; 25: 147-157.

Zhang W D et al. Inhibition of respiratory syncytial virus infection with intranasal siRNA nanoparticles targeting the viral NS1 gene. Nature Medicine 2005; 11: 56-62.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A drug delivery system comprising a thiolated chitosan nanoparticle wherein the thiol groups are cross-linked.

2. The drug delivery system according to claim 1 wherein the thiol groups are cross-linked by oxidation.

3. The drug delivery system according to claim 2 wherein the duration of the oxidation reaction linking the thiol groups is about 12 or less hours.

4. The drug delivery system according to claim 1 wherein the thiol groups are cross-linked by addition of one or more chemical reagents.

5. The drug delivery system according to claim 1 wherein the cross-linking of thiol groups is adapted to provide sustained release of one or more drugs.

6. The drug delivery system according to claim 1 wherein the thiolated chitosan particles range in molecular weight from about 10 kDa to about 100 kDa.

7. The drug delivery system according to claim 1 wherein the thiolated chitosan particles have a molecular weight of about 33 kDa.

8. The drug delivery system according to claim 1 wherein the thiolated chitosan particles are less than about 300 nm.

9. The drug delivery system according to claim 1 wherein the thiolated chitosan particles have a deacylation of about 90%.

10. A nucleic acid delivery system comprising a thiolated chitosan nanoparticle.

11. The nucleic acid delivery system according to claim 10 further comprising a nucleic acid molecule in association with the thiolated chitosan.

12. The nucleic acid delivery system according to claim 11 wherein the weight ratio of thiolated chitosan to nucleic acid is about 1:1 to about 10:1.

13. The nucleic acid delivery system according to claim 11 wherein the weight ratio of thiolated chitosan to nucleic acid is about 5:1 to about 10:1.

14. The nucleic acid delivery system according to claim 11 wherein the weight ratio of thiolated chitosan to nucleic acid is about 5:1.

15. The nucleic acid delivery system according to claim 10 wherein the thiolated chitosan nanoparticles are cross-linked.

16. The nucleic acid delivery system according to claim 15 wherein the cross-linking of thiol groups is adapted to provide sustained release of one or more nucleic acids.

17. The nucleic acid delivery system according to claim 15 wherein the thiol groups are crosslinked by oxidation.

18. The nucleic acid delivery system according to claim 17 wherein the duration of the oxidation reaction linking the thiol groups is about 12 or less hours.

19. The nucleic acid delivery system according to claim 15 wherein the thiol groups are cross-linked by addition of one or more chemical reagents.

20. The nucleic acid delivery system according to claim 10 wherein the thiolated chitosan particles range in molecular weight from about 10 kDa to about 100 kDa.

21. The nucleic acid delivery system of claim 10 wherein the thiolated chitosan particles have a molecular weight of about 33 kDa.

22. A method of delivering a nucleic acid to a cell comprising the steps of:
providing a thiolated chitosan nanoparticle;
providing a nucleic acid of interest;
combining the thiolated chitosan nanoparticle and the nucleic acid of interest under conditions sufficient to form nucleic acid-chitosan complexes; and
contacting a target cell with the nucleic acid-thiolated chitosan complex.

23. The method according to claim 22 further comprising the step of crosslinking the thiol residues of the thiolated chitosan nanoparticles.

24. The method according to claim 23 wherein the cross-linking of thiol groups is adapted to provide sustained release of one or more nucleic acids.

25. The method according to claim 23 wherein the crosslinking step is performed before the step of combining the thiolated chitosan nanoparticle and the nucleic acid of interest.

26. The method according to claim 23 wherein the thiol groups are cross-linked by oxidation.

27. The method according to claim 26 wherein the duration of the oxidation reaction linking the thiol groups is about 12 or less hours.

28. The method according to claim 23 wherein the thiol groups are cross-linked by addition of one or more chemical reagents.

29. The method according to claim 22 wherein the thiolated chitosan particles range in molecular weight from about 10 kDa to about 100 kDa.

30. The method according to claim 22 wherein the thiolated chitosan particles have a molecular weight of about 33 kDa.

31. The method according to claim 22 wherein the thiolated chitosan particles are less than about 300 nm.

32. A method of delivering a drug to a cell comprising the steps of:
providing one or more thiolated chitosan nanoparticles;
crosslinking the thiol residues of the one or more thiolated chitosan nanoparticles;
providing a drug of interest;
combining the thiolated chitosan nanoparticles and the drug of interest under conditions sufficient to form drug-thiolated chitosan complexes; and
contacting a target cell with the drug-crosslinked thiolated chitosan complex.

33. The method according to claim 32 wherein the cross-linking of thiol groups is adapted to provide sustained release of one or more drugs.

34. The method according to claim 32 wherein the thiol groups are cross-linked by oxidation.

35. The method according to claim 34 wherein the duration of the oxidation reaction linking the thiol groups is about 12 or less hours.

36. The method according to claim 32 wherein the thiol groups are cross-linked by addition of one or more chemical reagents.

37. The method according to claim 32 wherein the thiolated chitosan particles range in molecular weight from about 10 kDa to about 100 kDa.

38. The method according to claim 32 wherein the thiolated chitosan particles have a molecular weight of about 33 kDa.

39. The method according to claim 32 wherein the thiolated chitosan particles are less than about 300 nm.
